(19) United States
(12) Patent Application Publication
(10) Pub. No.: US 2021/0196111 A1
(43) Pub. Date: Jul. 1, 2021

(54) LIGHT-EMITTING DIODE BASED DIFFUSE OPTICAL SPECTROSCOPY TOOL

(71) Applicant: THE REGENTS OF THE UNIVERSITY OF CALIFORNIA, Oakland, CA (US)

(72) Inventors: Samir G. Shreim, Irvine, CA (US); Elliot L. Botvinick, Irvine, CA (US); Hamid R. Djalilian, Orange, CA (US); Sean Michael White, Irvine, CA (US); John Weidling, Irvine, CA (US)

(21) Appl. No.: 17/058,005

(22) PCT Filed: May 21, 2019
(86) PCT No.: PCT/US19/33393

§ 371 Date: Nov. 23, 2020

Related U.S. Application Data
(60) Provisional application No. 62/675,056, filed on May 22, 2018.

(51) Int. Cl. A61B 1/06; A61B 1/227; A61B 1/00

(52) U.S. Cl. CPC A61B 1/0638; A61B 1/00004; A61B 1/0684; A61B 1/227

(57) ABSTRACT

Systems and methods for use in detecting health condition of a physiological cavity or passageway are disclosed herein. In one example, the system may include one or more illuminators configured to illuminate a target area with light at discrete wavelengths. The system may additionally include detectors that are configured to receive a reflectance spectrum based on light emitted at discrete wavelengths from tissue and tissue constituents associated with the target area under analysis. Communicatively coupled to the one or more detectors, the processing unit is configured to analyse data associated with the reflectance spectra to produce one or more output values that identify the health condition.

### CROSS-REFERENCES TO RELATED APPLICATIONS

This application claims benefit of U.S. Patent Application No. 62/675,056, filed May 22, 2018, the specification(s) of which is/are incorporated herein in their entirety by reference.

### FIELD OF THE INVENTION

The present invention relates to the field of optical spectroscopy (OS). More particularly, the present invention relates to OS for assessment of health conditions of a patient, and in accordance with a specific embodiment of the disclosure, an LED-based OS tool for assessing the health condition of an ear.

### BACKGROUND

Otitis Media (OM), or middle ear inflammation, is the second most common cause for pediatrician office visits after the common cold. OM accounts for over 20 million office visits per year in the United States alone, with 75% of these being children under 3 years old. Acute Otitis Media (AOM) is a common, usually painful, type of OM that results from an abrupt onset infection for which antibiotics are usually prescribed. It is estimated that nearly two-thirds of children will experience AOM prior to their first birthday with more than 90% experiencing AOM prior to age 2. Approximately 80% of office visits result in a prescription for antibiotics, making suspected AOM the leading cause of antibiotic prescriptions. Despite this prevalence, AOM is one of the most commonly misdiagnosed conditions. For children younger than 1 year old, diagnostic certainty is a little better than one would expect by chance alone (58%) and improves only marginally for children up to 3.5 years (66%). This is problematic as more than three-quarters of all office visits relating to AOM are for children age 3 and younger, and thus, this misdiagnosed condition is responsible for nearly 5 billion dollars of unnecessary health costs per annum.

Poor diagnostic accuracy of AOM results from several factors. Firstly, the symptoms of AOM are numerous yet highly nonspecific. In a review of 43 clinical trials relating to AOM, 18 different sets of criteria were used to indicate AOM, with no more than 6 agreeing on the same set. In practice, a febrile (feverish) child with an earache who presents with a red (erythema) tympanic membrane (ear drum) on otoscopic examination (magnified visualization of the middle ear) will typically be prescribed antibiotics. Further, these symptoms are commonly exhibited in children with an elevated temperature due to a common cold. A major hurdle in accurate diagnosis of AOM during most ear examinations, especially one with an uncomfortable, crying toddler, is the presence of cerumen (ear wax) in an already very small ear canal. Cerumen not only obstructs view of the tympanic membrane, but removal can in itself cause redness. A busy clinician examining an uncooperative, crying young patient with exasperated parent(s) will often use any sign of redness in the ear as a justification for antibiotics. Taken together, it is not surprising that diagnosis of AOM is approximately as accurate as a coin flip. Even in older, presumably more cooperative children, diagnostic accuracy only rises to approximately 75%.

Despite these challenges, accurate diagnosis of OM is achievable. If the middle ear can be clearly visualized, then measurement of ear drum mobility (i.e. assessing presence of middle ear fluid), combined with visual assessment can improve diagnostic accuracy to nearly 100%. This can be accomplished by acoustic tympanometry, which requires an airtight seal between the otoscope and ear canal for ˜20 seconds, and pneumatic otoscopes that modulate pressure on the ear drum to assess its mobility, which is reduced if fluid is present (indicative of OM). Despite improved accuracy, these techniques are not widely used in primary care where many OM diagnoses occur because in practice, pneumatic otoscopy and acoustic tympanometry are difficult to accomplish on a resistant child, may be painful, and waxy ears confound both assessments and should be cleaned for accurate determination of OM.

Several clinical and home devices are also available to improve OM diagnosis. These include a hand-held device that assesses ear drum mobility using sound (EarCheck®), and several magnifying smartphone attachments (e.g. CellScope®). Smartphone attachments are generally intended to relay middle ear images to doctors for diagnosis. While image processing techniques for assessing OM on a smartphone have been demonstrated in laboratory settings, such algorithms have yet to replace a physician in commercial products. Importantly, while development and sale of smartphone attachments indicates market interest, they have not achieved widespread consumer or clinical adoption because they do not address the two challenges listed above. For example, EarCheck® and smartphone device accuracy is compromised when wax is present. Further, consumer cellphone-based technologies such as CellScope® require untrained users (i.e. parents) to acquire quality images, which is a challenge in upset children. Consequently, EarCheck® and CellScope® are prone to inaccurate diagnoses, erosion of user trust, limited adoption, and unreliable results.

### SUMMARY OF THE INVENTION

According to some aspects, the present invention features a non-invasive tool comprising one or more detectors configured to receive light and measure a reflectance intensity at predetermined wavelengths, and a processing unit communicatively coupled to the one or more detectors. The processing unit may comprise a memory that stores computer readable instructions that, during execution by the processing unit, causes the processing unit to receive signals from the one or more detectors, determine reflectance spectra associated with the signals received from the one or more detectors) at the predetermined wavelengths, generate data from the determined reflectance spectra, and conduct analytics on the data to determine a metric. In one embodiment, the predetermined wavelengths may range from about 400 nm to about 2000 nm. In another embodiment, the metric determines a health condition of a tissue being a source of the received light. The analytics being conducted on the data may determine whether the tissue, being a portion of the ear canal, is healthy or has serous or mucoid middle ear effusion, otitis media, otitis media with effusion, acute otitis media, otitis externa, cerumen impaction, or a foreign body.

According to other aspects, the present invention provides a method in which the non-invasive tool may be used. The method may comprise illuminating a target area with light, detecting reflected light at predetermined wavelengths from the illuminated target area, receiving signals corresponding to the reflected light at the predetermined wavelengths, determining reflectance spectra associated with the signals received at the predetermined wavelengths, generating data from the reflectance spectra, and conducting analytics on the data to determine a metric. In one example, the target area is the tissue and the method can determine a health condition of the tissue.

A practical application of an embodiment of the present invention is to provide one or more devices and methods that utilize reflected light in accordance with optical spectroscopy (OS) technologies to assess the health of the middle ear, as described herein. The device may be configured to illuminate the ear at specific wavelengths using multiple illuminators, collect reflected light using one or multiple detectors, and analyze reflected light and compute the reflectance spectra for diagnosing the health condition of the ear. Embodiments of the invention are given in the dependent claims. Different inventive aspects set forth in various embodiments of the invention can be freely combined with each other if they are not mutually exclusive.

In some aspects, an embodiment of the present invention discloses a compact, non-invasive tool for diagnosing ear conditions irrespective of presence of cerumen in an ear canal. In one embodiment, the tool may comprise a speculum configured to be positioned in the ear canal of a patient to visualize a portion of the ear cavity using visualization optics, and one or more spectral illumination assemblies configured to illuminate the ear canal with light at predetermined wavelengths, which are selected to diagnose specific ear conditions. The tool may additionally comprise one or more detector assemblies configured to receive light returning from the ear canal and measure a reflectance intensity as a function of the predetermined wavelengths. The light returning from the ear canal may comprise light that is reflected and scattered from tissue and tissue constituents of the ear canal. In some embodiments, the spectral illumination assemblies and the detector assemblies may be integrated within a housing that is flush with the speculum. The tool may additionally comprise a processing unit communicatively coupled to the one or more detector assemblies. The processing unit may comprise a memory that stores computer readable instructions that, when executed by the processing unit, causes the processing unit to control illumination and data acquisition, receive signals from the one or more detector assemblies, record reflectance signals received from the one or more detector assemblies at the predetermined wavelengths, generate data from the reflectance signals and analyse the data to determine a metric for the tissue and tissue constituents of the ear canal, and provide an indication based upon the metric, which is correlated to a health condition of the ear. The metric may be used to determine if the ear is healthy or if it has serous or mucoid middle ear effusion, otitis media, otitis media with effusion, acute otitis media, otitis externa, cerumen impaction, or a foreign body.

In some embodiments, the one or more spectral illumination assemblies may comprise one or more of light-emitting diode (LED) elements, laser diodes, or vertical-cavity surface-emitting laser (VCSELs). Each LED element, laser diode, laser diode or VCSEL may comprise a central wavelength matching with a specific wavelength of the predetermined wavelengths. In some embodiments, the one or more spectral illumination assemblies may comprise one or more filtered broadband light sources, such as white light sources. The filters for the filtered broadband sources may be selected based on the predetermined wavelengths. The predetermined wavelengths may be selected from a range of wavelengths of about 400 nm to about 2000 nm. In other embodiments, the one or more detector assemblies comprise photodiodes, photomultiplier tubes (PMT), complementary metal-oxide-semiconductor (CMOS) detectors, charge-coupled device (CCD) detector, spectrometers, spectrometers on a chip, spectroscopy sensors, and fabry-perot interferometers.

In some embodiments, the analysing may comprise comparing characteristics of the reflectance spectrum to one or more reference metric distributions. In one embodiment, the reference metric distribution may be determined based on machine learning or heuristics that considers data from one or more prior analysis of ear conditions. In another embodiment, the reference metric distributions may be stored locally in the memory of the processing unit or downloadable from a remote database.

In some embodiments, the tool may additionally include one or more Brightfield illumination assemblies configured to illuminate the ear canal for visual evaluation. In some embodiments, the tool may have optical diffusers in line each Brightfield illumination assembly for diffusing light from the Brightfield illumination assemblies into the ear canal. In other embodiments, the tool may include relay lenses integrated within the housing, wherein the relay lenses may be configured to propagate light from the one or more spectral illumination assemblies and the one or more Brightfield illumination assemblies into the ear canal. In yet other embodiments, the tool may additionally comprise visualization optics coupled to the housing wherein the visualization optics may be configured to visualize portions of the ear canal illuminated by the one or more spectral illumination assemblies and the one or more Brightfield illumination assemblies.

In some embodiments, the tool may further comprise a handle having a measure button protruding from an outer surface of the handle. In one aspect, the measure button may be operatively coupled to the processing unit. For example, upon activation of the measure button (e.g. via depressing the button), the processing unit executes computer-readable instructions stored in the memory, which causes the processing unit to: activate the one or more spectral illumination assemblies, one or more Brightfield illumination assemblies, and the one or more detector assemblies, and subsequent to the activation, simultaneously illuminate, detect, analyse and display the diagnostic result.

According to other aspects, the present invention features a method of detecting a health condition at least based on reflected light from tissue and tissue constituents in a passageway under diagnosis. In a non-limiting embodiment, the method may be applicable for detecting a health condition of an ear canal for example. The method may include providing an otoscope integrated with (i) an optical interface, (ii) a speculum, (iii) visualization optics, (iv) a dimmer switch, (v) a handle, and (vi) a measuring button. The optical interface may comprise one or more spectral illuminators configured to illuminate a portion of the ear canal at specific wavelengths, which are selected to detect otitis media (OM) in the ear, and one or more detector assemblies configured to receive light returning from the ear canal. Upon activation of the measuring button, the method may include serially illuminating each of the spectral illuminators at the specific wavelengths, simultaneously acquiring reflected light from tissue and tissue constituents in the ear canal using the one or more detector assemblies, and generating a reflectance spectrum based on signals acquired by the one or more detector assemblies. The reflectance spectrum may include intensity measurements at the specific wavelengths of illumination. The method may further include generating data from the reflectance spectrum, and analysing the data to assess characteristics of the reflectance spectrum and generate a diagnostic result that may identify if the ear is healthy or has OM.

It is to be understood that the detection methods described herein also has utility in other areas of diagnostics. Alternative to or in conjunction with the embodiments of the present invention, the non-invasive tool and methods may be used to conduct measurements as to the health of tissue within any physiological pathway or cavity including, but not limited to, the esophagus, skin, rectum, eye, nose, sinus, ureter, urethra, vagina, abdominal cavity, etc. Thus, the spectral approach can be combined with spatial information to indicate the presence or risk of various conditions in a spatial fashion, for example, diabetic ulcers, cancer, strep throat etc. In addition, because the above-described scheme enables optical miniaturization, the non-invasive tool can be built into a range of instruments besides otoscopes, for example any type of endoscope, microscope, or other visualization aid, etc.

In some embodiments, the photodiode assembly may comprise one or more photodiodes with distinct spectral sensitivities. In this way, one or more LEDs with spectral distributions overlapping each photodiode sensitivity may be simultaneously illuminated to reduce measurement time without crosstalk.

In other embodiments, the analysis may include applying a statistical learning model to the data, the statistical learning model comprising, but not limited to one or more of logit/probit models, Gaussian discriminant analysis, support vector machines, k-nearest neighbours, neural networks, Bayesian methods, and separation by inspection, or other parametric or non-parametric techniques. In some embodiments, the method may additionally include, after expiration of a specified period of time, resetting the otoscope and indicating to a user that the otoscope is ready to perform new measurements.

Non-limiting examples of the one or more spectral illuminators include light-emitting diode (LED) elements, laser diodes, vertical-cavity surface-emitting laser (VCSELs), or filtered broadband sources. In some embodiments, the specific wavelengths may be selected from a range of wavelengths ranging from about 400 nm to about 2000 nm. In other embodiments, the one or more detector assemblies may comprise photodiodes, photomultiplier tubes (PMT), complementary metal-oxide-semiconductor (CMOS) detectors, charge-coupled device (CCD) detectors, spectrometers, spectroscopy sensors and fabry-perot interferometers.

One of the unique and inventive technical features of the present invention is a system and analysis scheme that includes spectral determination of OM even in the presence of ear wax or cerumen, which overcomes a profound deficiency in the above-described conventional schemes. Another unique and inventive aspect of the present invention includes a formulation of logic (e.g., algorithm) that operates on only a limited set of wavelengths, which are provided by one or more illuminator assemblies, such as a small array of light-emitting diode (LED) light sources for example, to eliminate any requirement for full spectroscopic measurement (and a high-cost spectrometer unit, and problems associated with multi-colinearity). The set of wavelengths may be determined through testing and/or prior analyses. As an example, for an ear, one or more reference metric distributions may be formed to simulate the attributes of a patient with acute otitis media (AOM), otitis media effusion (OME), or a healthy ear. The reference metric distributions take into account one or more combinations of eardrum, erythema (redness), cerumen (wax) and middle ear fluids. Without wishing to limit the invention to any theory or mechanism, it is believed that the technical feature of the present invention advantageously provides for a compact, hand-held LED based non-invasive device to detect ear infection, through ear wax, if necessary for diagnosing otitis media. None of the presently known prior references or work has any of the unique inventive technical features of the present invention described herein.

In fact, the current thinking is that when diagnosing ear infections using spectroscopy, spectrometers or laser illumination are needed. As such, spectrometer measurements are absolute with respect to wavelength and this led to some disbelief that it would be possible to use LEDs for spectroscopy measurements since LEDs have a broad spectrum and additionally, the central wavelength of LEDs vary as part of normal manufacturing processes, drive current, and operating temperature. LED illumination spans a range of wavelengths centered on the design wavelength and this range is further unique to each LED and may be as small as +/−8 nm or as large as +/−60 nm. The net result is that spectra obtained using LEDs and a photodetector are considered to have far lower x-axis (i.e. wavelength) repeatability, reproducibility, and overall accuracy. In other words, a 490 nm LED may not always have a peak of 490 nm. If driven harder and hotter, the LED may run at 493 nm or 491 nm. Unexpectedly and surprisingly, the present invention not only effectively used LEDs for spectroscopy measurements, but also took advantage of the surprising discovery that the optical features indicating ear infection are broader than LED wavelength variability, which thereby enables use of LEDs for diagnosing ear infections in a cost-effective manner.

In the following description, numerous specific details are set forth. However, it is understood that embodiments of the disclosure may be practiced without these specific details. For instance, besides the middle ear, the system and method may be configured for use with assessing the health condition of other body parts including, but not limited to a throat, skin, body cavities accessed and/or imaged by endoscopy, or those exposed during surgery. In other instances, well-known structures and techniques have not been shown in detail in order not to obscure the understanding of this description. Those of ordinary skill in the art, with the included descriptions, will be able to implement appropriate functionality without undue experimentation.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIGS. 1A-1F illustrate non-limiting embodiments of a non-invasive tool for diagnosing a health condition of an ear. FIGS. 1A and 1B show an exterior view and an interior view of the tool, respectively. FIG. 1C is a detailed view of an optical head of the tool. FIG. 1D is a front view of the tool head. FIG. 1E is a detailed side view of the optical head and FIG. 1F is a detailed view of a spectral illumination assembly in the optical head, the spectral illumination assembly includes a multiLED chip.

FIGS. 2A-2B illustrates an exemplary embodiment of an algorithm using statistical learning to diagnose otitis media.

FIG. 3A shows the reflectance spectra obtained at discrete wavelengths from a healthy ear and an infected ear, wherein the reflectance spectra are acquired according to an embodiment of the invention.

FIG. 3B shows reflectance spectra from the healthy ear and the infected ear acquired using standard approach.

FIG. 4 shows a schematic diagram of the ear.

### DESCRIPTION OF PREFERRED EMBODIMENTS

Following is a list of elements corresponding to a particular element referred to herein:

100 tool

110 optical interface

112 spectroscopy unit

116 conical housing

120 speculum

130 visualization optics

135 dimmer switch

140 handle

142 outer surface of handle

145 measure button

147 otoscope head

150_{1-K} spectral illumination assemblies

152_{1-N} spectral illuminators

160_{1-L} Brightfield illumination assemblies

165 optical diffuser

170_{1-M} detector assemblies

180 relay lens

190 incident light

192 re-emitted light

302 reflectance plot from health ear

304 reflectance plot from infected ear

306 reflectance plot from health ear

308 reflectance plot from infected ear

400 ear

402 cerumen

404 erythema

406 fluid

408 middle ear cavity

410 tympanic membrane

412 ear canal

In the following description, numerous specific details are set forth. However, it is understood that embodiments of the disclosure may be practiced without these specific details. For instance, besides the middle ear, the system and method may be configured for use with assessing the health condition of other body parts including, but not limited to a throat, skin, body cavities accessed and/or imaged by endoscopy, or those exposed during surgery. In other instances, well-known structures and techniques have not been shown in detail in order not to obscure the understanding of this description. Those of ordinary skill in the art, with the included descriptions, will be able to implement appropriate functionality without undue experimentation.

References in the specification to “one embodiment” or “an embodiment,” may indicate that the embodiment described may include a particular feature, structure, or characteristic, but every embodiment may not necessarily include that particular feature, structure, or characteristic. Moreover, such phrases are not necessarily referring to the same embodiment. Further, when a particular feature, structure, or characteristic is described in connection with an embodiment, it is submitted that such feature, structure, or characteristic may be deployed in connection with other embodiments whether or not explicitly described.

In the following description, certain terminology is used to describe features of the invention. For example, in certain situations, the term “logic” may be representative of hardware and/or software that is configured to perform one or more functions. As hardware, the logic may include circuitry having data processing or storage functionality. Examples of such circuitry may include, but are not limited or restricted to a hardware processor (e.g., microprocessor with one or more processor cores, a digital signal processor, a programmable gate array, a microcontroller, an application specific integrated circuit “ASIC”, etc.), or even combinatorial elements.

Additionally, logic may be software in the form of one or more software modules, such as executable code in the form of an algorithm, an executable application, an application programming interface (API), a subroutine, a function, a procedure, an applet, a servlet, a routine, source code, object code, a shared library/dynamic load library, or one or more instructions. The software module(s) may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; semiconductor memory; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

Referring now to the figures, in one embodiment, the present invention features a non-invasive tool (100) comprising one or more spectral illumination assemblies (150_{1-K}) configured to illuminate a target area with light, and a detector assembly (170) comprising a spectrometer on a chip. The spectrometer on a chip may include an optical sensor configured to receive reflected light from the illuminated target area, and a processing unit communicatively coupled to the optical sensor and the one or more spectral illumination assemblies (150_{1-K}). The processing unit may comprise a processor operatively coupled to a memory that stores computer readable instructions that, upon execution by the processor, causes the processor to perform operations comprising controlling illumination of the one or more spectral illumination assemblies (150_{1-K}), receiving signals from the optical sensor, determining reflectance spectra associated with the signals received from the optical sensor at the predetermined wavelengths, generating data from the determined reflectance spectra, and conducting analytics on the data to determine a diagnostic metric. In one embodiment, the one or more spectral illumination assemblies (150_{1-K}) include a chip comprising one or more light emitting diodes. The predetermined wavelengths may be selected from a range of about 400 nm to about 2000 nm.

According to another embodiment, the non-invasive tool (100) may comprise one or more spectral illumination assemblies (150_{1-K}) configured to illuminate a target area with light, one or more detector assemblies (170_{1-M}) configured to receive light from the illuminated target area and measure a reflectance intensity at predetermined wavelengths ranging from about 400 nm to about 2000 nm, and a processing unit operatively coupled to the one or more detector assemblies (170_{1-M}). The processing unit may comprise a memory that stores computer readable instructions that, when executed by the processing unit, causes the processing unit to receive signals from the one or more detector assemblies, determine reflectance spectra from the signals received from the one or more detector assemblies (170_{1-M}) at the predetermined wavelengths, generate data from the reflectance spectra, and analyze the data to determine a diagnostic metric. In one embodiment, the one or more spectral illumination assemblies (150_{1-K}) include a chip comprising one or more light emitting diodes (LED), such as a chip-on-board LED.

In some embodiments, the non-invasive tool may be utilized in a method, for example, to determine a health condition. The method may comprise illuminating a target area with light using the one or more spectral illumination assemblies, detecting, from the illuminated target area, reflected light at predetermined wavelengths via the one or more detector assemblies, receiving, by the processing unit, signals corresponding to the reflected light at the predetermined wavelengths, determining, by the processing unit, reflectance spectra associated with the signals received at the predetermined wavelengths, generating, by the processing unit, data from the reflectance spectra, and conducting, by the processing unit, analytics on the data to determine a metric.

In accordance with the embodiments of present invention, the target area is a tissue and the diagnostic metric determines a health condition of the tissue being a source of the received light. The analytics being conducted on the data can determine whether the tissue, being a portion of the ear canal, is healthy or has serous or mucoid middle ear effusion, otitis media, otitis media with effusion, acute otitis media, otitis externa, cerumen impaction, or a foreign body.

According to some embodiments, the present invention discloses systems and methods that utilize reflected light in accordance with optical spectroscopy (OS) technologies to assess the health of the middle ear. The system may include a non-invasive tool featuring one or more spectral illumination assemblies that illuminate the ear canal with the help of relay optics at specified wavelengths. The system further includes logic that is configured to collect light returning to the system after interaction (e.g., scatter and/or absorption) with the matter accessible via the ear canal (e.g., portions of the ear canal, tympanic membrane, middle ear cavity, cerumen, air and/or fluid in the middle ear cavity, etc.) for use in detecting a health condition of the middle ear. This captured light, or “reflectance spectra,” is converted into data, which is analyzed to assess characteristics (e.g., magnitude) of the reflectance spectra. Herein reflectance spectra is used to produce a set of reflectance metrics, which may be compared with prior statistical distributions of the predetermined metrics (each directed to a different health condition for an ear) in order to classify the ear currently under analysis.

According to other embodiments, the system may be implemented in a method directed to (i) collecting light returning after interaction with matter inside and including the ear canal and middle ear cavity, and (ii) analyzing the characteristics (e.g. magnitude vs. wavelength) of the reflectance spectra associated with the collected light to identify the current health condition of the middle ear. Since each type of matter (feature) of the ear (e.g., tissue of the ear canal, tympanic membrane, air and/or fluid within the middle ear cavity, cerumen, etc.) may uniquely contribute to the magnitude of the reflectance spectra, this system is configured to more accurately diagnose different types of Otitis Media (OM), such as Acute Otitis Media (AOM) or Otitis Media with effusion (OME) for example, without requisite removal of cerumen (ear wax).

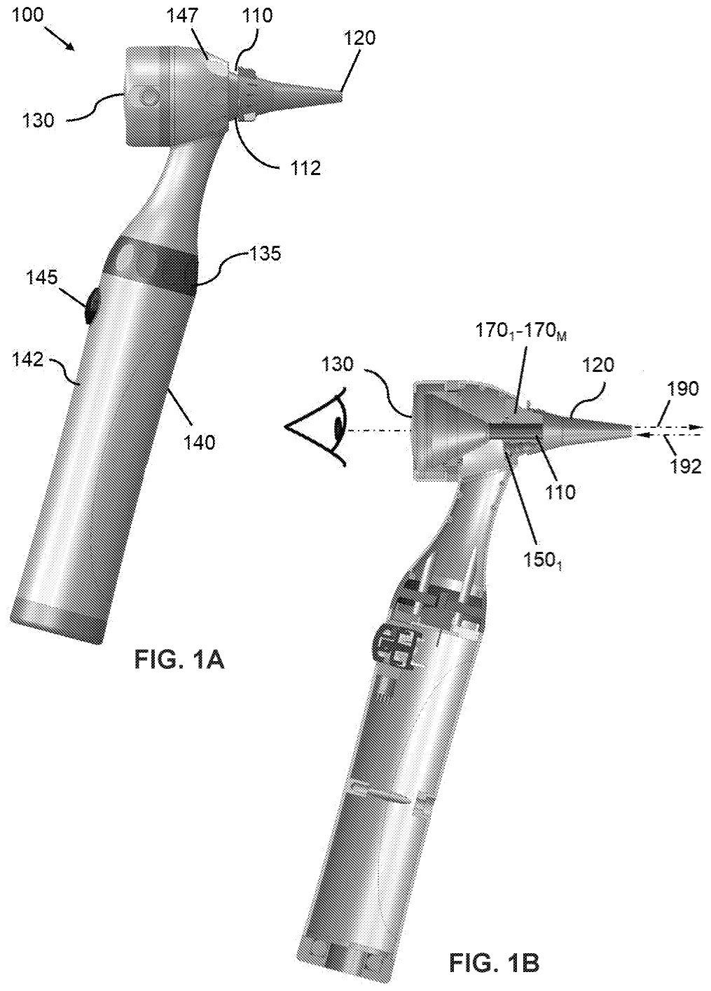

Referring to FIGS. 1A-1F, an exemplary embodiment of a non-invasive tool for diagnosing ear conditions using optical spectroscopy (OS)/reflectance spectroscopy is shown. As such, OS utilizes returned (e.g., reflected) light to detect tissue properties. Turning now to FIG. 1A, a non-invasive tool 100 for diagnosing ear conditions is shown. The non-invasive tool 100 may be adapted as an otoscope that comprises an otoscope head (147) attached to a handle (140) having a dimmer switch 135 and a measure button (145), and a speculum (120) attached to the otoscope head (147). In further embodiments, the otoscope head (147) may be integrated with an optical interface 110 and visualization optics 130. Coupled to the optical interface 110, the speculum 120 may be inserted into an ear canal of a patient to visualize a portion of the ear cavity using the visualization optics 130, such as a viewing window. Additionally, as shown in FIG. 1C, the optical interface 110 includes a spectroscopy unit 112 and relay lenses 180 that enable appropriate light propagation between the spectroscopy unit 112 and the portion of a body part (e.g. an ear cavity) under evaluation. With the visualization optics 130, the spectroscopy unit 112 and the relay optics 114 enable simultaneous illumination, detection and visualization of that portion of the ear cavity.

According to one embodiment, the dimmer switch 135 is positioned adjacent to the handle 140 to allow easy manipulation by the user. The dimmer switch 135 is used to activate the otoscope 100 and adjust the illumination level provided by the Brightfield illuminator assemblies described below. The measure button 145 may be situated to protrude from an outer surface 142 of the handle 140or positioned in any one of multiple ergonomic locations other than the outer surface 142 as shown. The measure button 145 activates the one or more illumination and detector assemblies described below.

Referring now to FIG. 1C, the optical interface 110 includes one or more spectral illumination assemblies 150₁-150_{K} (K≥1), one or more Brightfield illuminator assemblies 160₁-160_{L} (L≥1) to illuminate the ear cavity for visual inspection, and one or more detector assemblies 170₁-170_{M} (M≥1). The optical interface 110 may also include (i) one or more optical diffuser 165₁-165₂ oriented in-line with the Brightfield illuminator assemblies 160₁-160₂ to provide more uniform illumination and (ii) one or more relay lenses 180 to collect and relay light to and from the one or more illumination assemblies (e.g., spectral illumination assembly 150₁) and/or the one or more detector assemblies (e.g., detector assemblies 170₁-170₂). Herein, the illumination assemblies 150₁-150_{K} and the Brightfield illumination assemblies 160₁-160_{L} are implemented as different assemblies, although it is contemplated that the spectral illumination assemblies 150₁-150_{K} and the Brightfield illumination assemblies 160₁-160_{L} may be implemented as a single assembly, such as one or more spectral illuminators (e.g., spectral illuminator 152_{N} shown in FIG. 1F) within the illumination assembly 150₁ operate as a Brightfield illuminator by emitting white light for visual evaluation of the ear cavity through the visualization optics 130 and the speculum 120.

According to one embodiment of the invention, the optical interface 110 features a conical housing 116 that coordinates (i) the visual orientation provided by at least one Brightfield illuminator (not shown) deployed with the Brightfield illuminator assemblies 160₁-160_{L} and (ii) the light propagation paths emitted and re-emitted by one or more spectral illuminators implemented within the one or more spectral illumination assemblies 150₁-150_{K} (e.g., spectral illumination assembly 150₁) and one or more detectors (not shown) implemented within the one or more detector assemblies 170₁-170_{M}. Although illustrated in a conical form, the optical housing 116 may be designed in accordance with any structural representation, provided that such representation enables the one or more spectral illumination assemblies 150₁-150_{K} (e.g., spectral illumination assembly 150₁) to direct light to an ear cavity and enables the one or more detector assemblies 170₁-170_{M} to receive this re-emitted light from the ear cavity.

As shown in FIG. 1F, the spectral illumination assembly 150₁ includes one or more spectral illuminators 152₁-152_{N} (N≥1) operating as one or more light sources for spectroscopic evaluation of the portion of the ear cavity under evaluation. When using multiple spectral illuminators 152₁-152_{N} (N≥2), each of these spectral illuminators 152₁-152_{N} may be deployed as the same type of illuminator or may be deployed as different types of spectral illuminators. Examples of different types of spectral illuminators (e.g., spectral illuminator 152₁) may include, but are not limited or restricted to light emitting diodes (LEDs), laser diodes, broadband light sources, vertical-cavity surface-emitting laser (VCSELs), or any combination thereof. For instance, according to one embodiment, when deploying multiple spectral illuminators 152₁-152_{N}, each of the spectral illuminators 152₁-152_{N} may be deployed as LEDs each with a central wavelength within a range of 400-1700 nanometers.

Stated differently, an embodiment of the otoscope 100 may include one or more light sources with at least one of the one or more light sources specifically intended to be used with one or more photodetectors for spectral assessment of the ear. In one embodiment, a spectral illuminator assembly comprises one or more narrow band spectral illuminators such as LEDs, laser diodes, VCSELs, filtered broadband light or the like to measure reflectance in specific spectral bands. In this embodiment, the one or more spectral illuminators 152₁-152_{N} are illuminated individually using drive electronics located in the otoscope head 147 or in the handle 140 to measure ear reflectance in all desired spectral bands. These measurements comprise a dataset that can be compared to equations/metrics or historic distributions of data to assess the condition of the ear or other body parts (e.g. throat). In a second embodiment, the one or more spectral illuminators 152₁-152_{N} may be broadband (white light) and at least one or more photodetectors is configured to measure reflectance intensity versus wavelength. In one embodiment, the one or more detector assemblies 170₁-170_{M} may be implemented as a spectrometer to measure reflectance intensity versus wavelength including any type of miniaturized spectrometers such as a spectrometer sensor chip.

According to another embodiment, instead of discretizing emitted light 154 by the spectral illuminators 152₁-152_{N} with different central wavelengths within the above-identified wavelength range, the spectral illuminators 152₁-152_{N} may emit a broadband (or white) light, such as blackbody illumination for example, and the one or more detector assemblies 170₁-170_{M} may be configured to discretize the re-emitted light as received. It is further contemplated that the discretizing of the emitted light may be further accomplished through the use of filters (not shown) interposed between the spectral illuminators 152₁-152_{N} and the portion of the ear cavity under evaluation or the discretizing of the re-emitted light may be accomplished through the use of filters (not shown) interposed between the ear cavity under evaluation and the detector assemblies 170 illustrated in FIG. 1C.

Referring to FIG. 1C, each of the detector assemblies 170₁-170_{M} (e.g., detector assembly 170₁) may include one or more detectors for spectroscopic evaluation of the ear by receiving re-emitted light from the portion of the ear cavity under evaluation. The spectroscopic evaluation involves comparison of signal characteristics for example magnitude to heuristics calculated from previously collected data that identifies the health of the portion of the ear cavity under evaluation (e.g. otitis media, acute otitis media, or healthy conditions). Examples of a detector deployed within the detector assembly 170₁ may be an optically sensitive device, which may include, but is not limited or restricted to, a photodiode (e.g., silicon and/or InGaAs photodiode), avalanche photodiode, photomultiplier tube, complementary metal-oxide-semiconductor (CMOS) detector, charge-coupled device (CCD) detector, spectrometer (including a spectrometer on a chip), spectroscopy sensors, fabry-perot interferometer, or the like.

As shown in FIG. 1B, the otoscope 100 enables simultaneous visualization (through visualization optics 130 and optical interface 110) and spectroscopic measurement of the ear. Specifically, according to this embodiment, upon insertion of the speculum 120, which may be disposable, into the ear canal toward the middle ear cavity, one or more spectral illuminators 152₁-152_{N} within the illuminator assembly 1501 is activated to emit incident light 190 into the ear canal, and re-emitted light 192 is collected by one or more detectors within the detector assembly 170₁-170_{M}. In another embodiment, an optical waveguide (not shown) may be used as a replacement or in addition to one or more lenses 180. In another embodiment, an optical waveguide(s) (not shown) may be used to propagate light from illuminator assemblies 150₁-150_{N} to the portion of the ear cavity under evaluation and collect re-emitted light and relay it to detector assemblies 170₁-170_{M}. In another embodiment, visualization optics 130 may be used in addition to or replaced by one or more camera and display systems.

The data associated with the one or more detector assemblies 170₁-170_{M} may be processed internally within the otoscope 100 by a hardware processor, which may be deployed within the handle 140 and operates in accordance with a data analysis algorithm. According to this embodiment, all processing is performed locally. In another embodiment, the data may be processed externally from the otoscope 100. For instance, the data may be processed by a computer that is communicatively coupled to the otoscope 100. Additionally, or in the alternative, the data may be at least partially processed by a server (enterprise-based server or cloud-based server) that is in communication with the otoscope 100 via a network connection (e.g., local, enterprise network and/or public network such as the Internet).

Although not shown, in another embodiment of the disclosure, the non-invasive tool 100 may be integrated with one or more temperature sensing elements (e.g., thermopile, thermistor, thermocouple, etc.). This allows the non-invasive tool 100 to further detect the temperature of the patient. For example, temperature sensing element(s) may be integrated into a tip/end-effector/housing designed to accommodate ear canal shape.

In summary, the non-invasive tool 100 is configured to at least collect returning light (e.g., scattered, reflected light) and analyze signals arising from various ear conditions. Different features (matter) within the ear may constitute a set of optical contrasts, each contributing to the magnitude of the reflectance spectra. For example, hemoglobin species contribute to erythema (redness); lipid species constitute wax and create spectral differences pronounced but not limited to blue/green; water content of middle ear effusions create contrast in the infrared, etc. Hence, some or all features of the ear may contribute to reflectance by their optical properties (e.g. scattering and absorption). These constituents and thus optical properties vary in a consistent way with different types of ear pathology. In essence, waxy healthy, waxy infected, clean healthy, and clean infected ears each constitute populations with statistically separable reflectance spectra. The present invention collects information at only the wavelengths needed to make the distinction, avoiding redundant measurements. This leads to a more statistically robust measurement.

Additionally, to provide a more robust analysis, it is contemplated that legacy reflectance spectra (and/or calculated metrics or equations) may be stored in the non-transitory storage medium, deployed within the non-invasive tool 100 or within a remotely located analysis unit (e.g. a remote server, public cloud service, or a private cloud service, etc.) via the network connection, as metric reference distributions and compared metrics associated with subsequent reflectance spectra findings to improve both sensitivity, specificity, and accuracy or to obtain treatment suggestions for use in treating the diagnosed and/or indicated middle ear condition.

Optical spectroscopy (OS) utilizes returned (e.g., reflected) light to detect tissue properties. Light incident on turbid media, such as the eardrum or infected middle ear for example, is both absorbed and scattered. This creates a diffuse and unique chromatic spectra, which may be subsequently used as a spectral reference profile. Matter residing in the middle ear absorbs and scatters light in a unique way that may be assessed spectrally. Importantly, the diffuse nature of OS enables measurements in situations that preclude imaging, such as an ear canal occluded by wax. Provided the pathlength is long enough that transmitted or back-reflected light is diffused, the Beer-Lambert Law provides an accurate approximation to quantitatively determine the concentration of tissue chromophores:

I(λ)=I₀(λ)10^{-μ′}^{ₛ}^{(λ)l-(Σ}^{_{a,i}}^{(λ))l} Equation 1

where “I(λ)” is the measured light intensity as a function of wavelength λ, “I₀(λ)” is the incident intensity, “μ_{a,i}(λ)” is the i^{th} chromophore's absorption coefficient, “μ′ₛ(λ)” is the scattering coefficient and “l” is the optical pathlength.

Provided μ_{a,i}(λ) is known (e.g., oxy- and deoxyhemoglobin) and scattering and pathlength can be estimated, inversion of Equation (1) provides accurate, quantitative information on tissue properties. Inversion of Equation (1) may require at least as many wavelength measurements as chromophores, preferably many more. While sophisticated optics and signal processing make traditional OS expensive and bulky, the system and method of the present invention are designed with electronics that may be deployed in commercial clinical devices such as otoscopes, endoscopes, microscope, or other optical-based clinical devices.

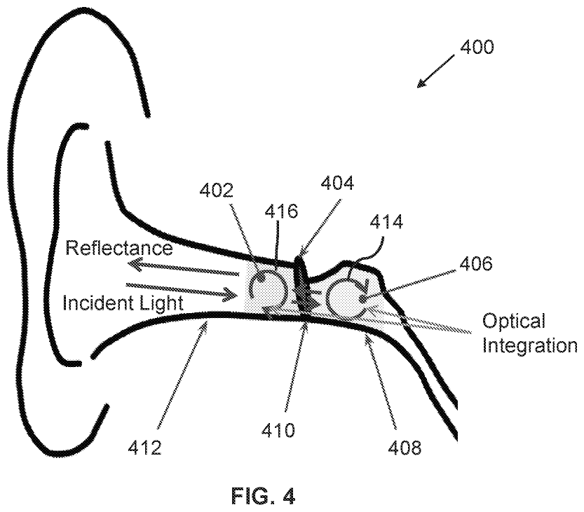

Turning now to FIG. 4, a schematic diagram of an ear 400 having an ear canal 412 is shown. The otoscope of the present invention may be used to illuminate matter accessible via the ear canal 412, especially the middle ear cavity 408. This matter may include tissue (e.g., portions of the ear canal 412, tympanic membrane 410, middle ear cavity 408, etc.) and tissue constituents (e.g., cerumen 402, erythema (redness) 404, bacteria, air or fluid 406 within the middle ear cavity, etc.). As previously described, it has been a challenge to diagnose conditions such as OM, and AOM, when there is cerumen 402 or ear wax present in the ear canal 412. Most of the currently available systems are unable to accurately determine the ear conditions in the presence of cerumen, hence either the ear wax has to be removed for accurate results, or the results are inaccurate if performed without removing the wax. However, the system of the present invention uses optical spectroscopy techniques to optically integrate signals reflected and scattered from the tissue constituents including cerumen, thereby allowing for a medical provider or user to consistently and accurately determine conditions such as OM even in the presence of cerumen.

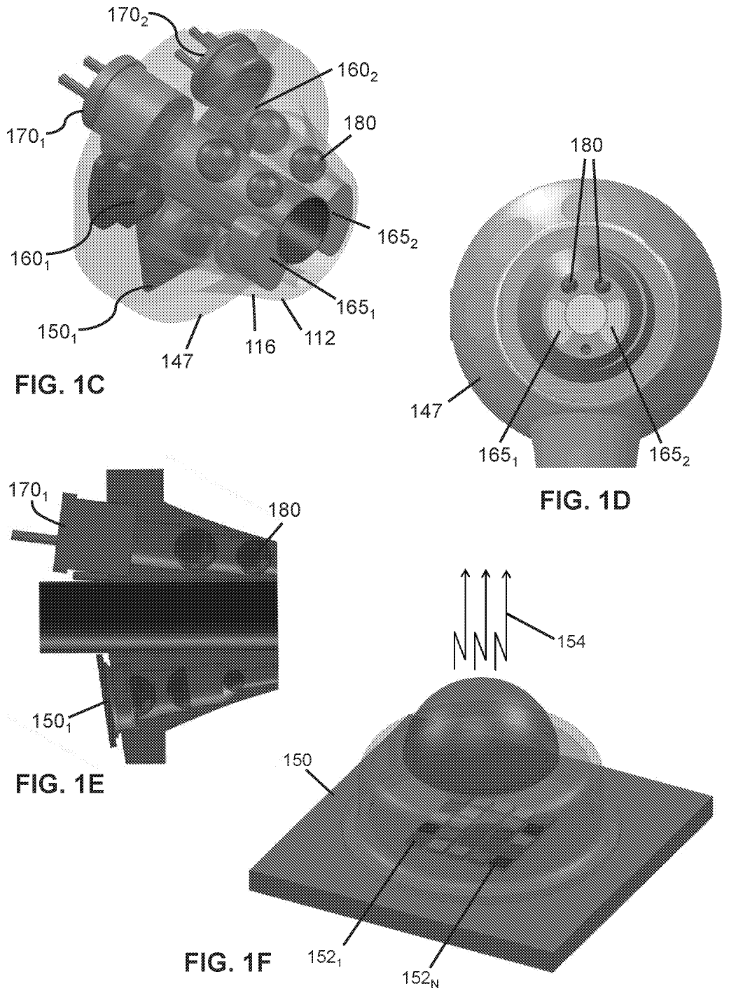

The system includes logic that is configured to collect light returning to the system after interaction (e.g., scatter and/or absorption) with the matter accessible via the ear canal (e.g., portions of the ear canal, tympanic membrane, middle ear cavity, cerumen, air and/or fluid in the middle ear cavity, etc.) for use in detecting a health condition of the middle ear. This captured light, sometimes referred to as the “reflectance spectra,” is converted into data, which is analyzed to assess characteristics (e.g., magnitude) of the reflectance spectra, as described with reference to FIGS. 2A and 2B, as described below.

According to some embodiments, the present invention provides a method of detecting a health condition of an ear. The method may comprise providing a tool (100), such as any one of the otoscope tools disclosed herein, inserting a portion of the speculum (120) within the ear canal, illuminating the ear canal with light at predetermined wavelengths via the spectral illumination assemblies (150_{1-K}), and detecting light returning from the ear canal, via the one or more detector assemblies (170_{1-M}). The light returning from the ear canal may comprise light that is reflected and scattered from tissue and tissue constituents of the ear canal. The method further comprises sending signals corresponding to the detected light from the one or more detector assemblies to the processing unit, generating diffuse reflectance spectra from the signals at the predetermined wavelengths, generating data from the reflectance spectra and analysing the data to determine a diagnostic metric for the tissue and tissue constituents of the ear canal, and generating a diagnostic result to identify the ear conditions based on the diagnostic metric. The method may be used to determine or diagnose if the patient has otitis media, acute otitis media, or if the ear is healthy, even if cerumen is present in the ear canal.

Referring to FIG. 2A, an illustrative embodiment of the operational flow of the otoscope 100 of FIG. 1A in use for detection of otitis media is shown. Initially, power is applied to the otoscope (200). Thereafter, the otoscope is configured to notify the user that it is ready to measure the condition of the portion of the ear cavity under evaluation and activates the one or more Brightfield illuminator assemblies implemented within the otoscope (205 and 210). The dimmer switch is utilized to adjust the intensity of the light produced by the Brightfield illuminator assemblies (215).

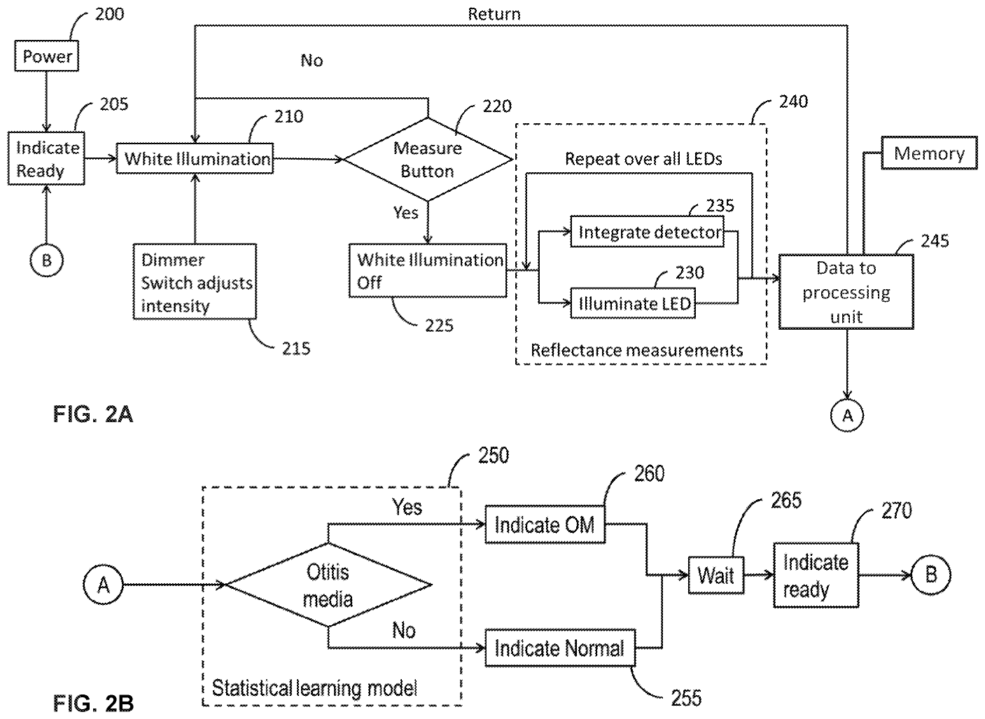

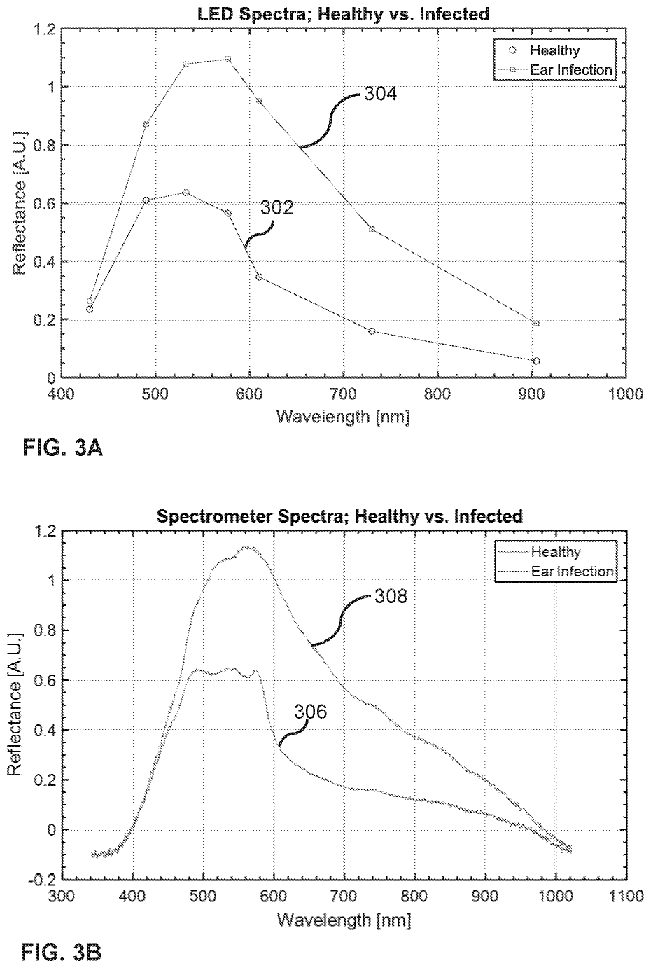

Upon activating (e.g. depressing) the measure button (220), the following operations occur in sequence: Brightfield illumination is turned off (225) and each of the spectral illuminators (e.g. LEDs, laser diodes, etc.) deployed within the spectral illumination assembly of the otoscope are serially illuminated (230) with signal acquisition by the one or more detector assemblies (235). The signal acquisition is performed concurrently (e.g. at least partially overlapping in time) with the spectral illumination and is repeated for each spectral illuminator (240). Such operations are referred to in FIG. 2A as reflectance measurements. The reflectance measurements produce data to process (245). As an illustrative example, the data to process may include voltages related to the intensity of re-emitted light originating from each spectral illuminator. Briefly, the reflectance measurements may be used to generate a reflectance spectra (as shown in FIG. 3A) that is used to produce a set of reflectance metrics, which may be compared with prior statistical distributions of the predetermined metrics (each directed to a different health condition for an ear) in order to classify the ear currently under analysis.

Similarly, the method features a plurality of operations for analyzing the reflectance spectra produced from the collected light. Such analysis of the reflectance spectra may include a comparison of certain characteristics of the reflectance spectra, such as the distribution of selected metrics, to one or more statistical distributions of metrics each directed to characteristics of different middle ear health conditions (hereinafter “reference metric distributions”). It is contemplated that each reference metric distribution may be based on machine learning or heuristics that considers data from one or more prior analyses of an ear condition. The reference metric distributions may be stored locally in the system or downloadable from a remote data store (e.g., cloud services) that operates as central storage for reference metric distributions (e.g., distributions shared by a practice group of physicians, a network of physicians who belong to a particular insurance network or hospital, or the like).

According to this embodiment, the system (non-invasive tool) and method are directed to (i) collecting light returning after interaction with matter inside and including the ear canal and middle ear cavity and (ii) analyzing the characteristics (e.g. magnitude vs. wavelength) of the reflectance spectra associated with the collected light to identify the current health condition of the middle ear. Since each type of matter (feature) of the ear (e.g., tissue of the ear canal, tympanic membrane, air and/or fluid within the middle ear cavity, cerumen, etc.) may uniquely contribute to the magnitude of the reflectance spectra, this system is configured to more accurately diagnose different types of OM, such as AOM or Otitis Media with effusion (OME) for example, without requisite removal of cerumen (ear wax).

According to some embodiments of the present invention, the reflectance spectra may be generated by illuminating the ear canal at specific wavelengths. As a non-limiting example, the ear canal may be illuminated at discrete wavelengths of about 420 nm, 480 nm, 540 nm, 580 nm, 620 nm, 740 nm, and 905 nm using the spectral illuminators. Herein, the values are for example purposes only, and are not meant to be limiting. It may be appreciated that the present invention recognizes that it is possible to capture the spectral information in terms of shape of the reflectance spectrum with fewer measurement points. As an example, consider plot 306 of FIG. 3B which shows reflectance spectra from healthy ear that is measured using a standard approach wherein the reflectance measurement is taken every 0.33 nm, which constitutes a total of 2047 measurements. In contrast, plot 302 of FIG. 3A which shows reflectance spectra from a healthy ear that is taken at strategic wavelengths has fewer measurement points, but still shows similar reflectance profile to plot 306. Though plot 302 of FIG. 3A has only 7 measurement points, it is still able to capture the spectral information (given by the overall shape profile of the plot) of the healthy ear. Likewise, compare plots 304 and 308 of FIGS. 3A and 3B, respectively. Even with only 7 measurements, the plot 304 is able to capture the reflectance profile of infected ear. Thus, the present invention discloses acquiring the reflectance measurements at fewer points, thereby greatly reducing the time and expense required for making such measurement, while also improving numerical stability and generalizability of classification algorithms used to diagnose health conditions.

In one embodiment, the spectral illuminators may include an array of light emitting elements, which may be selected such that their central wavelength coincides with the selected wavelengths. These specific wavelengths may be selected based on one or more of absorption, reflectance, and scattering of the tissue and the tissue constituents, for example. Specific LEDs may be selected to measure redness, waxiness, water content, scattering, or other empirical optical features.

It is contemplated that the detector assembly may operate in according to a number detection patterns. As a first example, the detector assembly may reset with each new spectral illumination conducted by a single spectral illuminator or a plurality of spectral illuminators operating together. As another example, the detector assembly may collect re-emitted light continuously as spectral illuminator(s) are cycled. As yet another example, the Brightfield illumination may intersperse spectral illumination to give the perception that Brightfield illumination is continuously being applied, although Brightfield illumination is temporarily turned off during spectral illumination, or Brightfield illumination may be continuously applied and the resultant difference in measured signal (e.g. voltage difference or voltage rate) subtracted from the data to process.

Referring to FIG. 2B, an illustrative embodiment of the statistical operations performed by the otoscope 100 (or device handling the data processing) is shown. Herein, a statistical learning model is applied to the data to process from the detector assembly to compute a diagnostic metric based on statistical analyses of the data to process (e.g., metrics determined from the reflectance spectra) versus previously collected data (e.g., reference metric distribution) as shown in item 250. This is achieved, at least in whole or in part, by any number of statistical classification methods/routines such as, for example, logit/probit models, Gaussian (using for example linear/ellipsoidal, boundaries or those drawn by inspection) discriminant analysis, support vector machines, k-nearest neighbors, neural networks, Bayesian methods, separation by inspection, other parametric or non-parametric methods or any combination thereof.

Based on the computed diagnostic metric, the statistical learning model generates a diagnostic result that identifies whether the portion of the ear cavity under evaluation is healthy or not (items 255 and 260). After a specified period of time has elapsed the otoscope resets and indicates to the user that it is ready to perform another measurement as described in FIG. 2A (items 265 and 270).

Herein, according to one embodiment, the statistical learning model is implemented as a software module stored in the above-described, non-transitory storage medium and accessible by the hardware processor, which is positioned within the housing of the non-invasive tool 100 (e.g., otoscope, endoscope, etc.). According to another embodiment, the statistical learning model may be deployed as logic within the non-invasive tool 100 of the invention, such as the tool shown in FIGS. 1A-1F.

While the invention has been described in terms of several embodiments, those skilled in the art will recognize that the invention is not limited to the embodiments described, and can be practiced with modification and alteration within the spirit and scope of the appended claims. While the scheme is advantageous in the diagnosis of ear conditions, it is contemplated that this approach also has utility in other areas of diagnostics. For instance, alternative to being used to analyze the health of an ear canal, the non-invasive tool may be used to conduct measurements as to the health of tissue within any physiological pathway including, but not limited to, the esophagus, skin, rectum, eye, nose, sinus, ureter, urethra, vagina, abdominal cavity, etc. For example, the canal is an attractive site for pulse oximetry. A reflectance based approach enables measurements at shorter wavelengths than traditional pulse oximetry where the contrasts of oxy-hemoglobin and deoxy-hemoglobin are particularly strong. In another example, the non-invasive tool may be applied to the back of the throat or skin could be used to indicate infection, inflammation, cancer, or perhaps other health conditions. Using an imaging (for example, CMOS or CCD array detector, the spectral approach can be combined with spatial information to indicate the presence or risk of various conditions in a spatial fashion, for example, diabetic ulcers, cancer, etc. In addition, because the above-described scheme enables optical miniaturization, the non-invasive tool can be built into a range of instruments besides otoscopes, for example any type of endoscope, microscope, or other visualization aid, etc. The description is thus to be regarded as illustrative instead of limiting.

As used herein, the term “about” refers to plus or minus 10% of the reference point, such as a prescribed wavelength of reflected light.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the below claims and in the figures are solely for ease of examination of this patent application, and are exemplary, and are not intended in any way to limit the scope of the claims to the particular features having the corresponding reference numbers in the drawings. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

1.-38. (canceled)

39. A non-invasive tool (100) comprising:
a. a head portion (147) comprising:
i. one or more spectral illumination assemblies (150_{1-K}) configured to illuminate a target area with light including a set of predetermined wavelengths, wherein the one or more spectral illumination assemblies (150_{1-K}) includes a chip comprising one or more light emitting diodes; and
ii. one or more detector assemblies (170_{1-M}) configured to receive light from the illuminated target area and measure a reflectance intensity at predetermined wavelengths ranging from about 400 nm to about 2000 nm; and
b. a processing unit operatively coupled to the one or more detector assemblies (170_{1-M}), the processing unit comprising a memory that stores computer readable instructions that, when executed by the processing unit, causes the processing unit to:
i. determine reflectance spectra from signals received from the one or more detector assemblies (170_{1-M}) at the predetermined wavelengths;
ii. generate data from the reflectance spectra; and
iii. analyze the data to determine a diagnostic metric.

40. The non-invasive tool (100) of claim 39, wherein the target area is a tissue, wherein the diagnostic metric determines a health condition of the tissue being a source of the received light.

41. The non-invasive tool (100) of claim 39, wherein the processing unit is configured to analyze the data by at least determining whether the tissue, being a portion of the ear canal, is healthy or has serous or mucoid middle ear effusion, an acute infection, cerumen impaction, or a foreign body.

42. The non-invasive tool (100) of claim 39, wherein the one or more light emitting diodes comprises a plurality of light emitting diodes assembled as part of an integrated circuit (IC) chip each emitting a light signal of a different central wavelength that is part of the set of predetermined wavelengths.

43. The non-invasive tool (100) of claim 39, wherein the head portion corresponds to an otoscope head configured to house (i) the one or more spectral illumination assemblies (150_{1-K}) configured to illuminate the target area being an ear canal with the light at the predetermined wavelengths, and (ii) the one or more detector assemblies (170_{1-M}) configured to receive light returning from the ear canal, wherein the light returning from the ear canal comprises light that is reflected and scattered from tissue and tissue constituents of the ear canal

44. The non-invasive tool (100) of claim 39, wherein the head portion (147) corresponds to an otoscope head further comprising one or more Brightfield illumination assemblies (160_{1-L)}, the one or more Brightfield illumination assemblies (160_{1-L}) are configured to illuminate the target area being an ear canal for visual evaluation.

45. The non-invasive tool (100) of claim 44, wherein the otoscope head (147) further comprising one or more optical diffusers (165), each optical diffuser is paired to and collinear with a corresponding Brightfield illumination assembly of the one or more Brightfield illumination assemblies (160_{1-L}) so that at least a first optical diffuser of the one or more optical diffuser is configured to diffuse light from the corresponding Brightfield illumination assembly into the ear canal.

46. The non-invasive tool (100) of claim 44 further comprising an optical waveguide or relay lenses (180) integrated within the otoscope head (147), wherein either the optical waveguide is or the relay lenses are configured to propagate light from the one or more spectral illumination assemblies (150_{1-K}) and the one or more Brightfield illumination assemblies (160_{1-L}) into the ear canal.

47. The non-invasive tool (100) of claim 44 further comprising visualization optics (130) disposed in the otoscope head (147), wherein the visualization optics (130) is configured to visualize portions of the ear canal illuminated by the one or more spectral illumination assemblies (150_{1-K}) and the one or more Brightfield illumination assemblies (160_{1-L})

48. The non-invasive tool (100) of claim 44 further comprising a handle (140) coupled to the otoscope head (147), wherein the handle (140) has a measure button (145) disposed on an outer surface (142) of the handle, wherein the measure button (145) is configured to activate the one or more spectral illumination assemblies (150_{1-K}), one or more Brightfield illumination assemblies (160_{1-L}), and the one or more detector assemblies (170_{1-M}).

49. The non-invasive tool (100) of claim 39, wherein the one or more spectral illumination assemblies (150_{1-K}) comprise one or more of light-emitting diode (LED) elements, multi-LED chips, laser diodes, or vertical-cavity surface-emitting laser (VCSELs), and wherein each LED element, laser diode, laser diode or VCSEL comprises a central wavelength matching with a specific wavelength of the predetermined wavelengths.

50. The non-invasive tool (100) of claim 39, wherein the one or more detector assemblies (170_{1-M}) comprise (i) photodiodes, (ii) complementary metal-oxide-semiconductor (CMOS) detectors, or (iii) spectroscopy sensors.

51. A method comprising:
a. illuminating a target area with light at different predetermined wavelengths, wherein the light is emitted by a plurality of light emitting diodes;
b. detecting reflected light from the illuminated target area;
c. receiving signals corresponding to the reflected light at the predetermined wavelengths;
d. determining reflectance spectra associated with the signals received at the predetermined wavelengths;
e. generating data from the reflectance spectra; and
f. conducting analytics on the data to determine a metric.

52. The method of claim 51, wherein the conducting of the analytics on the data comprises comparing characteristics of the reflectance spectra to one or more reference metric distributions, wherein each reference metric distribution is determined based on machine learning or heuristics that considers data from one or more prior analysis of ear conditions.

53. The method of claim 52, wherein the reference metric distributions are stored locally in a memory of a processing unit that controls operations (a)-(f) or downloadable from a remote database.

54. The method of claim 51, wherein the conducting of the analytics on the data comprises applying a statistical learning model to the data, the statistical learning model comprising one or more of (i) logit/probit models, (ii) Gaussian discriminant analysis, (iii) support vector machines, (iv) k-nearest neighbours, (v) neural networks, (vi) Bayesian methods, or (vii) separation by inspection.

55. A non-invasive tool (100) for diagnosing ear conditions irrespective of a presence of cerumen in an ear canal, the tool comprising:
a. a speculum (120) configured to be positioned in the ear canal of a patient to visualize a portion of the ear cavity using visualization optics (130);
b. one or more spectral illumination assemblies (150_{1-K}) configured to illuminate the ear canal with light at predetermined wavelengths, the predetermined wavelengths selected to diagnose specific ear conditions;
c. one or more detector assemblies (170_{1-M}) configured to receive light returning from the ear canal and measure a reflectance intensity as a function of the predetermined wavelengths, the light returning from the ear canal comprising light that is reflected and scattered from tissue and tissue constituents of the ear canal, wherein the spectral illumination assemblies and the detector assemblies are integrated within a housing (116) that is flush with the speculum (120);
d. a processing unit operatively coupled to the one or more detector assemblies (170_{1-M}), the processing unit comprising a memory that stores computer readable instructions that, when executed by the processing unit, causes the processing unit to:
i. control illumination and data acquisition;
ii. receive signals from the one or more detector assemblies;
iii. record reflectance signals received from the one or more detector assemblies (170_{1-M}) at the predetermined wavelengths;
iv. generate data from the reflectance signals and analyse the data to determine a metric for the tissue and tissue constituents of the ear canal; and
v. provide a diagnostic result based on the metric, which is correlated to a health condition of the ear;
wherein the diagnostic result is generated even if cerumen in the ear canal is present, wherein the health condition of the ear is healthy or it has serous or mucoid middle ear effusion, otitis media, otitis media with effusion, acute otitis media, otitis externa, cerumen impaction, or a foreign body.

56. The non-invasive tool (100) of claim 55 further comprising one or more Brightfield illumination assemblies (160_{1-L}) configured to illuminate the ear canal for visual evaluation and one or more optical diffusers (165), wherein one optical diffuser is in line with one Brightfield illumination assembly, wherein the one or more optical diffusers (165) are configured to diffuse light from the Brightfield illumination assemblies (160_{1-L}) into the ear canal.

57. The non-invasive tool (100) of claim 56 further comprising relay lenses (180) and/or optical waveguides integrated within the housing (116), wherein the relay lenses and/or optical waveguides are configured to propagate light from the one or more spectral illumination assemblies (150_{1-K}) and the one or more Brightfield illumination assemblies (160_{1-L}) into the ear canal.

58. The non-invasive tool (100) of claim 56, wherein the visualization optics (130) is configured to visualize portions of the ear canal illuminated by the one or more spectral illumination assemblies (150_{1-K}) and the one or more Brightfield illumination assemblies (160_{1-L}).
